SEQUENCE LISTING (24 sequences)

SEQ ID NO: 1 - protein, 374 aa, Homo sapiens
  misc_feature: GENBANK Accession no. M80632
MARSLTWGCCPWCLTEEEKTAARIDQEINRILLEQKKQEREELKLLLLGPGESGKSTFIKQMRIIHGVGYSEEDRRAFRLLIYQNIFVSMQAMIDAMDRLQIPFSRPDSKQHASLVMTQDPYKVSTFEKPYAVAMQYLWRDAGIRACYERRREFHLLDSAVYYLSHLERISEDSYIPTAQDVLRSRMPTTGINEYCFSVKKTKLRIVDVGGQRSERRKWIHCFENVIALIYLASLSEYDQCLEENDQENRMEESLALFSTILELPWFKSTSVILFLNKTDILEDKIHTSHLATYFPSFQGPRRDAEAAKSFILDMYARVYASCAEPQDGGRKGSRARRFFAHFTCATDTQSVRSVFKDVRDSVLARYLDEINLL

SEQ ID NO: 2 - protein, 374 aa, Homo sapiens
  misc_feature: GENBANK Accession no. M63904
MARSLTWRCCPWCLTEDEKAAARVDQEINRILLEQKKQDRGELKLLLLGPGESGKSTFIKQMRIIHGAGYSEEERKGFRPLVYQNIFVSMRAMIEAMERLQIPFSRPESKHHASLVMSQDPYKVTTFEKRYAAAMQWLWRDAGIRACYERRREFHLLDSAVYYLSHLERITEEGYVPTAQDVLRSRMPTTGINEYCFSVQKTNLRIVDVGGQKSERKKWIHCFENVIALIYLASLSEYDQCLEENNQENRMKESLALFGTILELPWFKSTSVILFLNKTDILEEKIPTSHLATYFPSFQGPKQDAEAAKRFILDMYTRMYTGCVDGPEGSKKGARSRRLFSHYTCATDTQNIRKVFKDVRDSVLARYLDEINLL

SEQ ID NO: 3 - protein, 355 aa, Homo sapiens
  misc_feature: GENBANK Accession no. NM_002073
MGCRQSSEEKEAARRSRRIDRHLRSESQRQRREIKLLLLGTSNSGKSTIVKQMKIIHSGGFNLEACKEYKPLIIYNAIDSLTRIIRALAALRIDFHNPDRAYDAVQLFALTGPAESKGEITPELLGVMRRLWADPGAQACFSRSSEYHLEDNAAYYLNDLERIAAADYIPTVEDILRSRDMTTGIVENKFTFKELTFKMVDVGGQRSERKKWIHCFEGVTAIIFCVELSGYDLKLYEDNQTSRMAESLRLFDSICNNNWFINTSLILFLNKKDLLAEKIRRIPLTICFPEYKGQNTYEEAAVYIQRQFEDLNRNKETKEIYSHFTCATDTSNIQFVFDAVTDVIIQNNLKYIGLC

SEQ ID NO: 4 - protein, 374 aa, Artificial
  Synthetic
MARSLTWRCCPWCLTEDEKAAARVDQEINRILLEQKKQDRGELKLLLLGPGESGKSTFIKQMRIIHGAGYSEEERKGFRPLVYQNIFVSMRAMIEAMERLQIPFSRPESKHHASLVMSQDPYKVTTFEKRYAAAMQWLWRDAGIRACYERRREFHLLDSAVYYLSHLERITEEGYVPTAQDVLRSRMPTTGINEYCFSVQKTNLRIVDVGGQKSERKKWIHCFENVIALIYLASLSEYDQCLEENNQENRMKESLALFGTILELPWFKSTSVILFLNKTDILEEKIPTSHLATYFPSFQGPKQDAEAAKRFILDMYTRMYTGCVDGPEGSNRNKETKEIYSHFTCATDTSNIQFVFDAVTDVIIQNNLKYIGLC

SEQ ID NO: 5 - protein, 858 aa, Mus musculus
MPALAIMGLSLAAFLELGMGASLCLSQQFKAQGDYILGGLFPLGSTEEATLNQRTQPNSIPCNRFSPLGLFLAMAMKMAVEEINNGSALLPGLRLGYDLFDTCSEPVVTMKSSLMFLAKVGSQSIAAYCNYTQYQPRVLAVIGPHSSELALITGKFFSFFLMPQVSYSASMDRLSDRETFPSFFRTVPSDRVQLQAVVTLLQNFSWNWVAALGSDDDYGREGLSIFSSLANARGICIAHEGLVPQHDTSGQQLGKVLDVLRQVNQSKVQVVVLFASARAVYSLFSYSIHHGLSPKVWVASESWLTSDLVMTLPNIARVGTVLGFLQRGALLPEFSHYVETHLALAADPAFCASLNAELDLEEHVMGQRCPRCDDIMLQNLSSGLLQNLSAGQLHHQIFATYAAVYSVAQALHNTLQCNVSHCHVSEHVLPWQLLENMYNMSFHARDLTLQFDAEGNVDMEYDLKMWVWQSPTPVLHTVGTFNGTLQLQQSKMYWPGNQVPVSQCSRQCKDGQVRRVKGFHSCCYDCVDCKAGSYRKHPDDFTCTPCNQDQWSPEKSTACLPRRPKFLAWGEPVVLSLLLLLCLVLGLALAALGLSVHHWDSPLVQASGGSQFCFGLICLGLFCLSVLLFPGRPSSASCLAQQPMAHLPLTGCLSTLFLQAAETFVESELPLSWANWLCSYLRGLWAWLVVLLATFVEAALCAWYLNAFPPEVVTDWSVLPTEVLEHCHVRSWVSLGLVHITNAMLAFLCFLGTFLVQSQPGRYNRARGLTFAMLAYFITWVSFVPLLANVQVAYQPAVQMGAILVCALGILVTFHLPKCYVLLWLPKLNTQEFFLGRNAKKAADENSGGGEAAQGHNE

SEQ ID NO: 6 - DNA, 1353 bp, Mus musculus
  misc_feature: GENBANK Accession no. M80632
caggccctgtgatgtcacctggtggtctgtgaagcgcccaccatggcccggtccctgacttggggctgctgtccctggtgcctgacagaggaggagaagactgccgccagaatcgaccaggagatcaacaggattttgttggaacagaaaaaacaagagcgcgaggaattgaaactcctgctgttggggcctggtgagagcgggaagagtacgttcatcaagcagatgcgcatcattcacggtgtgggctactcggaggaggaccgcagagccttccggctgctcatctaccagaacatcttcgtctccatgcaggccatgatagatgcgatggaccggctgcagatccccttcagcaggcctgacagcaagcagcacgccagcctagtgatgacccaggacccctataaagtgagcacattcgagaagccatatgcagtggccatgcagtacctgtggcgggacgcgggcatccgtgcatgctacgagcgaaggcgtgaattccaccttctggactccgcggtgtattacctgtcacacctggagcgcatatcagaggacagctacatccccactgcgcaagacgtgctgcgcagtcgcatgcccaccacaggcatcaatgagtactgcttctccgtgaagaaaaccaaactgcgcatcgtggatgttggtggccagaggtcagagcgtaggaaatggattcactgtttcgagaacgtgattgccctcatctacctggcctccctgagcgagtatgaccagtgcctagaggagaacgatcaggagaaccgcatggaggagagtctcgctctgttcagcacgatcctagagctgccctggttcaagagcacctcggtcatcctcttcctcaacaagacggacatcctggaagataagattcacacctcccacctggccacatacttccccagcttccagggaccccggcgagacgcagaggccgccaagagcttcatcttggacatgtatgcgcgcgtgtacgcgagctgcgcagagccccaggacggtggcaggaaaggctcccgcgcgcgccgcttcttcgcacacttcacctgtgccacggacacgcaaagcgtccgcagcgtgttcaaggacgtgcgggactcggtgctggcccggtacctggacgagatcaacctgctgtgacgcgggacagggaaccccaagcgcgacgcgtcgtggcgaggacatacctccccctggtggccgcgcgtggaactgcaggtccaggagctgccaagtggggaagccagcccacaggagagagtcctgcttctactgggccccaagccagctcctgtaattattcctcgccttctctagtgttggaag

SEQ ID NO: 7 - DNA, 29 bp, Artificial
  Synthetic
cgcaagctttctgtgaagcgcccaccatg

SEQ ID NO: 8 - DNA, 37 bp, Artificial
  Synthetic
gcattacgatgcggccgcgcgtcacagcaggttgatc

SEQ ID NO: 9 - DNA, 2060 bp, Homo sapiens
  misc_feature: GENBANK Accession no. M63904
tgttcccagcactcaagccttgccaccgccgagccgggcttcctgggtgtttcaggcaaggaagtctaggtccctggggggtgacccccaaggaaaaggcagcctccctgcgcacccggttgcccggagccctctccagggccggctgggctgggggttgccctggccagcaggggcccgggggcgatgccacccggtgccgactgaggccaccgcaccatggcccgctcgctgacctggcgctgctgcccctggtgcctgacggaggatgagaaggccgccgcccgggtggaccaggagatcaacaggatcctcttggagcagaagaagcaggaccgcggggagctgaagctgctgcttttgggcccaggcgagagcgggaagagcaccttcatcaagcagatgcggatcatccacggcgccggctactcggaggaggagcgcaagggcttccggcccctggtctaccagaacatcttcgtgtccatgcgggccatgatcgaggccatggagcggctgcagattccattcagcaggcccgagagcaagcaccacgctagcctggtcatgagccaggacccctataaagtgaccacgtttgagaagcgctacgctgcggccatgcagtggctgtggagggatgccggcatccgggcctgctatgagcgtcggcgggaattccacctgctcgattcagccgtgtactacctgtcccacctggagcgcatcaccgaggagggctacgtccccacagctcaggacgtgctccgcagccgcatgcccaccactggcatcaacgagtactgcttctccgtgcagaaaaccaacctgcggatcgtggacgtcgggggccagaagtcagagcgtaagaaatggatccattgtttcgagaacgtgatcgccctcatctacctggcctcactgagtgaatacgaccagtgcctggaggagaacaaccaggagaaccgcatgaaggagagcctcgcattgtttgggactatcctggaactaccctggttcaaaagcacatccgtcatcctctttctcaacaaaaccgacatcctggaggagaaaatccccacctcccacctggctacctatttccccagtttccagggccctaagcaggatgctgaggcagccaagaggttcatcctggacatgtacacgaggatgtacaccgggtgcgtggacggccccgagggcagcaagaagggcgcacgatcccgacgccttttcagccactacacatgtgccacagacacacagaacatccgcaaggtcttcaaggacgtgcgggactcggtgctcgcccgctacctggacgagatcaacctgctgtgacccaggccccacctggggcaggcggcaccggcgggcgggtgggaggtgggagtggctgcagggaccctagtgtcctggtctatctctccagcctcggcccacacgcaagggagtcgggggacggcccgctgctggccgctctcttctctgcctctcaccaggacagccgccccccagggtactcctgcccttgcttgactcagtttccctcctttgaaagggaaggagcaaaacggccatttgggatgccagggtggatgaaaaggtgaagaaatcaggggattgagacttgggtgggtgggcatctctcaggagccccatctccgggcgtgtcacctcctgggcagggttctgggaccctctgtgggtgacgcacaccctgggatggggctagtagagccttcaggcgccttcgggcgtggactctggcgcactctagtggacaggagaaggaacgccttccaggaacctgtggactaggggtgcagggacttccctttgcaaggggtaacagaccgctggaaaacactgtcactttcagagctcggtggctcacagcgtgtcctgccccggtttgcggacgagagaaatcgcggcccacaagcatcccccatcccttgcaggctgggggctgggcatgctgcatcttaaccttttgtatttattccctcaccttctgcagggctccgtgcgggctgaaattaaagatttcttag

SEQ ID NO: 10 - DNA, 2679 bp, Homo sapiens
  misc_feature: GENBANK Accession no. NM_002073
gagaccaggaccatgggatgtcggcaaagctcagaggaaaaagaagcagcccggcggtcccggagaattgaccgccacctgcgctcagagagccagcggcaacgccgcgaaatcaagctgctcctgctgggcaccagcaactcaggcaagagcaccatcgtcaaacagatgaagatcatccacagcggcggcttcaacctggaggcctgcaaggagtacaagcccctcatcatctacaatgccatcgactcgctgacccgcatcatccgggccctggccgccctcaggatcgacttccacaaccccgaccgcgcctacgacgctgtgcagctctttgcgctgacgggccccgctgagagcaagggcgagatcacacccgagctgctgggtgtcatgcgacggctctgggccgacccaggggcacaggcctgcttcagccgctccagcgagtaccacctggaggacaacgcggcctactacctgaacgacctggagcgcatcgccgcagctgactatatccccactgtcgaggacatcctgcgctcccgggacatgaccacgggcattgtggagaacaagttcaccttcaaggagctcaccttcaagatggtggacgtgggggggcagaggtcagagcgcaaaaagtggatccactgcttcgagggcgtcacagccatcatcttctgtgtggagctcagcggctacgacctgaaactctacgaggataaccagacaagtcggatggcagagagcttgcgcctctttgactccatctgcaacaacaactggttcatcaacacctcactcatcctcttcctgaacaagaaggacctgctggcagagaagatccgccgcatcccgctcaccatctgctttcccgagtacaagggccagaacacgtacgaggaggccgctgtctacatccagcggcagtttgaagacctgaaccgcaacaaggagaccaaggagatctactcccacttcacctgcgccaccgacaccagtaacatccagtttgtcttcgacgcggtgacagacgtcatcatacagaacaatctcaagtacattggcctttgctgaggagctgggcccggggcgcctgcctatggtgaaacccacggggtgtcatgccccaacgcgtgctagagaggcccaatccaggggcagaaaacagggggcctaaagaatgtcccccaccccttggcctctgcctccttggccccacatttctgcaaacataaatatttacggatagattgctaggtagatagacacacacacatgcacacacacacatctggagatggcaaaatcctctaaaatgtcgaggtctcttgaagacttgagaagctgtcacaaggtcactacaagcccaacctgccccttcactttgccttcctgagttggccccactccacttgggggtctgcattggattgttagggataggcagcagggctgaggcaaggtaggccaactgcacccctgtcacctggaggagggccggctcgctgcccgagctctggcctagggaccttgccgctgaccaagagggaggaccagtgcagggtctgtgcaccttccctgctggcctgcacacagctgctcagcaccatttcattctggacctgggaccttaggagccgggtgacagcactaaccagacctccagccactcacagctctttttaaaaaacagcttcaaaatatgcagcaaaaaccaatacaacaaaacgagtggcacgatttatttcaaactaggccagctgggattccagcttttcttctactagtctgatgttttataaatcaaaacctggttttccttctctggcatttttttttgttttttgttttttggttttttttttttttttggccaaatctcgtggtgtttcgcagaaaaaaatccagaaaatttcaaatgcagttgagtattcttttttaaatgcagattttcaaaacatattttttttcaggtggtcttttttgtgtctggcttgctgagtgtaaaagttgttatctggacgatctgtctctctgctccaaagaaattttggagtgagtggcagtcctgcgccagcctcgcgggacacgtgttgtacataagcctctgcagtgtcctcttgttaatggtggggttttctgctttgtttttatttaagaaaataaacacgacatatttaaagaaggttctttcacctgggagcaaatgaacaatagctaagtgtcttggtatttaaagagtaaattatttgtggctttgctgagtgaaggaaggggagcaaggggtggtgcccctggtcccagcatgccccgcgcctgagactggctggaaatgctctgactcctgtgaaggcacagccagcgttgtggcctgagggaggccctgctgggaccctgatctgggccttcctgtcccagggcctatgggcaactgcgttgaaaggacgttcgccaagggccgtgtgtaaatacgaactgcgccatggagaggagaggcactgccggagcccttgccagatctccctccctctctctgtgcagtagctgtgtgtccgaggtcagtgtgcggaatcacagccaaggacgtgaagagatgtacgggggaaagagaagctggggattggatgaaagtcaaaggttgtctactttaagaaaataaaataccctgaatgg

SEQ ID NO: 11 - DNA, 29 bp, Artificial
  Synthetic
cgcaagcttgactgaggccaccgcaccat

SEQ ID NO: 12 - DNA, 29 bp, Artificial
  Synthetic
ctccttgtttcggttgctgccctcggggc

SEQ ID NO: 13 - DNA, 30 bp, Artificial
  Synthetic
ggccccgagggcagcaaccgaaacaaggag

SEQ ID NO: 14 - DNA, 37 bp, Artificial
  Synthetic
gcattacgatgcggccgcagctcctcagcaaaggcca

SEQ ID NO: 15 - DNA, 1122 bp, Artificial
  Synthetic
atggcccgctcgctgacctggcgctgctgcccctggtgcctgacggaggatgagaaggccgccgcccgggtggaccaggagatcaacaggatcctcttggagcagaagaagcaggaccgcggggagctgaagctgctgcttttgggcccaggcgagagcgggaagagcaccttcatcaagcagatgcggatcatccacggcgccggctactcggaggaggagcgcaagggcttccggcccctggtctaccagaacatcttcgtgtccatgcgggccatgatcgaggccatggagcggctgcagattccattcagcaggcccgagagcaagcaccacgctagcctggtcatgagccaggacccctataaagtgaccacgtttgagaagcgctacgctgcggccatgcagtggctgtggagggatgccggcatccgggcctgctatgagcgtcggcgggaattccacctgctcgattcagccgtgtactacctgtcccacctggagcgcatcaccgaggagggctacgtccccacagctcaggacgtgctccgcagccgcatgcccaccactggcatcaacgagtactgcttctccgtgcagaaaaccaacctgcggatcgtggacgtcgggggccagaagtcagagcgtaagaaatggatccattgtttcgagaacgtgatcgccctcatctacctggcctcactgagtgaatacgaccagtgcctggaggagaacaaccaggagaaccgcatgaaggagagcctcgcattgtttgggactatcctggaactaccctggttcaaaagcacatccgtcatcctctttctcaacaaaaccgacatcctggaggagaaaatccccacctcccacctggctacctatttccccagtttccagggccctaagcaggatgctgaggcagccaagaggttcatcctggacatgtacacgaggatgtacaccgggtgcgtggacggccccgagggcagcaaccgcaacaaggagaccaaggagatctactcccacttcacctgcgccaccgacaccagtaacatccagtttgtcttcgacgcggtgacagacgtcatcatacagaacaatctcaagtacattggcctttgc

SEQ ID NO: 16 - DNA, 2529 bp, Mus musculus
  misc_feature: GENBANK Accession no. AY032622
atgcttttctgggcagctcacctgctgctcagcctgcagctggccgttgcttactgctgggctttcagctgccaaaggacagaatcctctccaggtttcagcctccctggggacttcctcctggcaggcctgttctccctccatgctgactgtctgcaggtgagacacagacctctggtgacaagttgtgacaggtctgacagcttcaacggccatggctatcacctcttccaagccatgcggttcaccgttgaggagataaacaactccacagctctgcttcccaacatcaccctggggtatgaactgtatgacgtgtgctcagagtcttccaatgtctatgccaccctgagggtgcccgcccagcaagggacaggccacctagagatgcagagagatcttcgcaaccactcctccaaggtggtggcactcattgggcctgataacactgaccacgctgtcaccactgctgccctgctgagcccttttctgatgcccctggtcagctatgaggcgagcagcgtgatcctcagtgggaagcgcaagttcccgtccttcttgcgcaccatccccagcgataagtaccaggtggaagtcatagtgcggctgctgcagagcttcggctgggtctggatctcgctcgttggcagctatggtgactacgggcagctgggcgtacaggcgctggaggagctggccactccacggggcatctgcgtcgccttcaaggacgtggtgcctctctccgcccaggcgggtgacccaaggatgcagcgcatgatgctgcgtctggctcgagccaggaccaccgtggtcgtggtcttctctaaccggcacctggctggagtgttcttcaggtctgtggtgctggccaacctgactggcaaagtgtggatcgcctccgaagactgggccatctccacgtacatcaccaatgtgcccgggatccagggcattgggacggtgctgggggtggccatccagcagagacaagtccctggcctgaaggagtttgaagagtcctatgtccaggcagtgatgggtgctcccagaacttgcccagaggggtcctggtgcggcactaaccagctgtgcagggagtgtcacgctttcacgacatggaacatgcccgagcttggagccttctccatgagcgctgcctacaatgtgtatgaggctgtgtatgctgtggcccacggcctccaccagctcctgggatgtacctctgggacctgtgccagaggcccagtctacccctggcagcttcttcagcagatctacaaggtgaatttccttctacataagaagactgtagcattcgatgacaagggggaccctctaggttattatgacatcatcgcctgggactggaatggacctgaatggacctttgaggtcattggttctgcctcactgtctccagttcatctagacataaataagacaaaaatccagtggcacgggaagaacaatcaggtgcctgtgtcagtgtgtaccagggactgtctcgaagggcaccacaggttggtcatgggttcccaccactgctgcttcgagtgcatgccctgtgaagctgggacatttctcaacacgagtgagcttcacacctgccagccttgtggaacagaagaatgggcccctgaggggagctcagcctgcttctcacgcaccgtggagttcttggggtggcatgaacccatctctttggtgctattagcagctaacacgctattgctgctgctgctgattgggactgctggcctgtttgcctggcgtcttcacacgcctgttgtgaggtcagctgggggtaggctgtgcttcctcatgctgggttccttggtagctgggagttgcagcctctacagcttcttcgggaagcccacggtgcccgcgtgcttgctgcgtcagcccctcttttctctcgggtttgccattttcctctcctgtctgacaatccgctccttccaactggtcatcatcttcaagttttctaccaaggtacccacattctaccacacttgggcccaaaaccatggtgccggaatattcgtcattgtcagctccacggtccatttgttcctctgtctcacgtggcttgcaatgtggaccccacggcccaccagggagtaccagcgcttcccccatctggtgattcttgagtgcacagaggtcaactctgtgggcttcctggtggctttcgcacacaacatcctcctctccatcagcacctttgtctgcagctacctgggtaaggaactgccggagaactataacgaagccaaatgtgtcaccttcagcctgctcctccacttcgtatcctggatcgctttcttcaccatgtccagcatttaccagggcagctacctacccgcggtcaatgtgctggcagggctggccactctgagtggcggcttcagcggctatttcctccctaaatgctacgtgattctctgccgtccagaactcaacaacacagaacactttcaggcctccatccaggactacacgaggcgctgcggcactacctga

SEQ ID NO: 17 - DNA, 2532 bp, Mus musculus
  misc_feature: GENBANK Accession No. AY032623
atgggaccccaggcgaggacactccatttgctgtttctcctgctgcatgctctgcctaagccagtcatgctggtagggaactccgactttcacctggctggggactacctcctgggtggcctctttaccctccatgccaacgtgaagagtgtctctcacctcagctacctgcaggtgcccaagtgcaatgagtacaacatgaaggtgttgggctacaacctcatgcaggccatgcgattcgccgtggaggaaatcaacaactgtagctctttgctgcccggcgtgctgctcggctacgagatggtggatgtctgctacctctccaacaatatccagcctgggctctacttcctgtcacagatagatgacttcctgcccatcctcaaagactacagccagtacaggccccaagtggtggctgttattggcccagacaactctgagtctgccatcaccgtgtccaacattctctcctacttcctcgtgccacaggtcacatatagcgccatcaccgacaagctgcaagacaagcggcgcttccctgccatgctgcgcactgtgcccagcgccacccaccacatcgaggccatggtgcaactgatggttcacttccagtggaactggatcgtggtgctggtgagcgatgacgattatggccgagagaacagccacctgctgagccagcgtctgaccaacactggcgacatctgcattgccttccaggaggttctgcccgtaccagaacccaaccaggctgtgaggcctgaggagcaggaccaactggacaacatcctggacaagctgcggcggacttcggcgcgtgtggtggtgatattctcgccggagctgagcctgcacaacttcttccgtgaggtgctgcgctggaacttcacgggctttgtgtggattgcctctgagtcctgggccatcgaccctgttctacacaacctcacagagctgcgccacacgggcactttcctgggtgtcaccatccagagggtgtccatccctggcttcagccagttccgagtgcgccatgacaagccagggtatcgcatgcctaacgagaccagcctgcggactacctgtaaccaggactgcgacgcctgcatgaacatcactgagtccttcaacaacgttctcatgctttcgggggagcgtgtggtctacagcgtgtactcggccgtctacgcggtggcccacaccctccacagactcctccactgcaatcaggtccgctgcaccaagcaaatcgtctatccatggcagctactcagggagatctggcatgtcaacttcacgctcctgggcaaccagctcttcttcgacgaacaaggggacatgccgatgctcctggacatcatccagtggcagtggggcctgagccagaaccccttccaaagcatcgcctcctactcccccaccgagacgaggctgacctacattagcaatgtgtcctggtacacccccaacaacacggtccccatatccatgtgttctaagagttgccagcctgggcaaatgaaaaaacccataggcctccacccatgctgcttcgagtgtgtggactgtccgccggacacctacctcaaccgatcagtagatgagtttaactgtctgtcctgcccgggttccatgtggtcttacaagaacaacatcgcttgcttcaagcggcggctggccttcctggagtggcacgaagtgcccactatcgtggtgaccatcctggccgccctgggcttcatcagtacgctggccattctgctcatcttctggagacatttccagacgcccatggtgcgctcggcgggcggccccatgtgcttcctgatgctggtgcccctgctgctggcgttcgggatggtccccgtgtatgtgggcccccccacggtcttctcctgtttctgccgccaggctttcttcaccgtttgcttctccgtctgcctctcctgcatcacggtgcgctccttccagattgtgtgcgtcttcaagatggccagacgcctgccaagcgcctacggtttctggatgcgttaccacgggccctacgtcttcgtggccttcatcacggccgtcaaggtggccctggtggcgggcaacatgctggccaccaccatcaaccccattggccggaccgaccccgatgaccccaatatcataatcctctcctgccaccctaactaccgcaacgggctactcttcaacaccagcatggacttgctgctgtccgtgctgggtttcagcttcgcgtacgtgggcaaggaactgcccaccaactacaacgaagccaagttcatcaccctcagcatgaccttctccttcacctcctccatctccctctgcacgttcatgtctgtccacgatggcgtgctggtcaccatcatggatctcctggtcactgtgctcaactttctggccatcggcttggggtactttggccccaaatgttacatgatccttttctacccggagcgcaacacttcagcttatttcaatagcatgattcagggctacacgatgaggaagagctag

SEQ ID NO: 18 - DNA, 2577 bp, Mus musculus
  CDS (1)..(2574)
atgccagctttggctatcatgggtctcagcctggctgctttcctggagcttgggatgggggcctctttgtgtctgtcacagcaattcaaggcacaaggggactacatactgggcgggctatttcccctgggctcaaccgaggaggccactctcaaccagagaacacaacccaacagcatcccgtgcaacaggttctcaccccttggtttgttcctggccatggctatgaagatggctgtggaggagatcaacaatggatctgccttgctccctgggctgcggctgggctatgacctatttgacacatgctccgagccagtggtcaccatgaaatccagtctcatgttcctggccaaggtgggcagtcaaagcattgctgcctactgcaactacacacagtaccaaccccgtgtgctggctgtcatcggcccccactcatcagagcttgccctcattacaggcaagttcttcagcttcttcctcatgccacaggtcagctatagtgccagcatggatcggctaagtgaccgggaaacgtttccatccttcttccgcacagtgcccagtgaccgggtgcagctgcaggcagttgtgactctgttgcagaacttcagctggaactgggtggccgccttagggagtgatgatgactatggccgggaaggtctgagcatcttttctagtctggccaatgcacgaggtatctgcatcgcacatgagggcctggtgccacaacatgacactagtggccaacagttgggcaaggtgctggatgtactacgccaagtgaaccaaagtaaagtacaagtggtggtgctgtttgcctctgcccgtgctgtctactccctttttagttacagcatccatcatggcctctcacccaaggtatgggtggccagtgagtcttggctgacatctgacctggtcatgacacttcccaatattgcccgtgtgggcactgtgcttgggtttttgcagcggggtgccctactgcctgaattttcccattatgtggagactcaccttgccctggccgctgacccagcattctgtgcctcactgaatgcggagttggatctggaggaacatgtgatggggcaacgctgtccacggtgtgacgacatcatgctgcagaacctatcatctgggctgttgcagaacctatcagctgggcaattgcaccaccaaatatttgcaacctatgcagctgtgtacagtgtggctcaagcccttcacaacaccctacagtgcaatgtctcacattgccacgtatcagaacatgttctaccctggcagctcctggagaacatgtacaatatgagtttccatgctcgagacttgacactacagtttgatgctgaagggaatgtagacatggaatatgacctgaagatgtgggtgtggcagagccctacacctgtattacatactgtgggcaccttcaacggcacccttcagctgcagcagtctaaaatgtactggccaggcaaccaggtgccagtctcccagtgttcccgccagtgcaaagatggccaggttcgccgagtaaagggctttcattcctgctgctatgactgcgtggactgcaaggcgggcagctaccggaagcatccagatgacttcacctgtactccatgtaaccaggaccagtggtccccagagaaaagcacagcctgcttacctcgcaggcccaagtttctggcttggggggagccagttgtgctgtcactcctcctgctgctttgcctggtgctgggtctagcactggctgctctggggctctctgtccaccactgggacagccctcttgtccaggcctcaggtggctcacagttctgctttggcctgatctgcctaggcctcttctgcctcagtgtccttctgttcccagggcggccaagctctgccagctgccttgcacaacaaccaatggctcacctccctctcacaggctgcctgagcacactcttcctgcaagcagctgagacctttgtggagtctgagctgccactgagctgggcaaactggctatgcagctaccttcggggactctgggcctggctagtggtactgttggccacttttgtggaggcagcactatgtgcctggtatttgaacgctttcccaccagaggtggtgacagactggtcagtgctgcccacagaggtactggagcactgccacgtgcgttcctgggtcagcctgggcttggtgcacatcaccaatgcaatgttagctttcctctgctttctgggcactttcctggtacagagccagcctggccgctacaaccgtgcccgtggtctcaccttcgccatgctagcttatttcatcacctgggtctcttttgtgcccctcctggccaatgtgcaggtggcctaccagccagctgtgcagatgggtgctatcctagtctgtgccctgggcatcctggtcaccttccacctgcccaagtgctatgtgcttctttggctgccaaagctcaacacccaggagttcttcctgggaaggaatgccaagaaagcagcagatgagaacagtggcggtggtgaggcagctcagggacacaatgaatga

SEQ ID NO: 19 - DNA, 29 bp, Artificial
  Synthetic
ggaattcatgcttttctgggcagctcacc

SEQ ID NO: 20 - DNA, 38 bp, Artificial
  Synthetic
gcattacgatgcggccgctcaggtagtgccgcagcgcc

SEQ ID NO: 21 - DNA, 27 bp, Artificial
  Synthetic
ggaattcatgggaccccaggcgaggac

SEQ ID NO: 22 - DNA, 40 bp, Artificial
  Synthetic
gcattacgatgcggccgcctagctcttcctcatcgtgtag

SEQ ID NO: 23 - DNA, 29 bp, Artificial
  Synthetic
ggaattcatgccagctttggctatcatgg

SEQ ID NO: 24 - DNA, 41 bp, Artificial
  Synthetic
gcattacgatgcggccgctcattcattgtgttcctgagctg

(19) United States
(12) Patent Application Publication
(10) Pub. No.: US 2005/0048508 A1
(43) Pub. Date: Mar. 3, 2005

(54) Trehalose receptor and method for detecting trehalose with the same

(75) Inventors: Toshio Ariyasu, Okayama (JP); Shuji Matsumoto, Okayama (JP); Fumiyo Kyono, Okayama (JP); Toshiharu Hanaya, Okayama (JP); Shigeyuki Arai, Okayama (JP); Masao Ikeda, Okayama (JP); Masashi Kurimoto, Okayama (JP)

Correspondence Address: BROWDY AND NEIMARK, P.L.L.C., 624 Ninth Street, N.W., Washington, DC, 20001-5303, US

(73) Assignee: KABUSHIKI KAISHA HAYASHIBARA SEIBUTSU KAGAKU KENKYUJO, Okayama-shi (JP)

(21) Appl. No.: 10/663,650

(22) Filed: Sep. 17, 2003

(30) Foreign Application Priority Data
Nov. 5, 2002 (JP) 321887/2002

(51) Int. Cl. C12Q 1/68; G01N 33/53; G01N 33/567; C07H 21/04; C07K 14/705

(52) U.S. Cl. 435/6; 435/7.2; 435/69.1; 435/325; 435/320.1; 530/350; 536/23.5

(57) ABSTRACT

Disclosed are a mammalian trehalose receptor which comprises a protein comprising any of the amino acid sequences of SEQ ID NOs: 1, 2, 3 and 5; or a protein comprising any of the amino acid sequences of SEQ ID NOs: 1, 4 and 5; and a method for detecting trehalose using an animal cell in which the trehalose receptor has been expressed.

### BACKGROUND OF THE INVENTION

1. Field of the Invention

The present invention relates to a mammalian trehalose receptor and a method for detecting trehalose using the same.

2. Description of the Prior Art

The establishment of technology of producing trehalose from material starches has enabled to produce trehalose at a lesser cost and to distribute food products and cosmetics containing trehalose in the market. Recently, from a viewpoint of caring consumers, the disclosure of data for ingredients, contained in food products and cosmetics, has been required; accordingly, there needed is a method for quantitatively detecting trehalose, contained in such food products and cosmetics, accurately and easily to objectively reconfirm the accuracy of the trehalose content specified on the labels of these products. Examples of conventionally proposed methods for detecting trehalose include the one, disclosed in Journal of the Japanese Society for Food Science and Technology, Vol. 45, No. 6, pp. 381-384 (1998), i.e., a method for detecting trehalose comprising the steps of extracting saccharides including trehalose, trimethylsilylating the extracted saccharides, and separating trehalose from the extracted saccharides to quantify the separated trehalose on gas chromatography. The above method is applicable to accurately quantify trehalose in food products on the order of ppm, however, it has, as a demerit, a complicated handling that it inevitably requires the steps of extracting and purifying saccharides from a test sample and trimethylsilylating the saccharides. Therefore, a simpler method has been required.

The fact that trehalose, with a 45% sweetening power of sucrose, can be tasted by the tongue leads to an estimation that it would be sensed by the taste cells in the taste buds of the tongue, suggesting the presence of a receptor of trehalose (hereinafter designated as “trehalose receptor”, unless specified otherwise). The use of such a receptor would facilitate the detection of trehalose, however, there has not yet been known the existence of any trehalose receptor in mammals including humans. As disclosed in Science, Vol. 289, pp. 116-119 (2000), a trehalose receptor was cloned from fruit-fly (Drosophila). Based on a finding by the present inventors, a trial of cloning the gene of a mRNA, prepared from a mouse tongue tissue using the DNA sequence of the above trehalose receptor, resulted in failure of finding any protein in mammals such as mice, that corresponds to the protein of trehalose receptor expressed in the fruit-fly. Nature, Vol. 413, No. 13, pp. 211-225 (2001) reveals different receptors in terms of gustatory sensation, such as sucrose receptors. For example, Cell, Vol. 106, pp. 381-390 (2001) discloses a hetero dimmer of T1R2 and T1R3 proteins as sucrose receptors; and Nature, Vol. 416, No. 14, pp. 199-202 (2002) discloses a hetero dimmer of T1R1 and T1R3 proteins as L-amino acid receptors. In addition, Cell, Vol. 106, pp. 381-390 (2001) discloses that α15, α16, and αZ proteins, as G protein α-subunits, correlate to the reaction of the above sweet taste receptors. However, these reports never suggest a trehalose receptor.

### SUMMARY OF THE INVENTION

The present invention was made based on the above-mentioned circumstances and it aims to reveal a trehalose receptor, and a method for detecting trehalose (may be abbreviated as “trehalose detection method”, hereinafter) in a test sample directly and easily using the receptor without requiring any extraction and purification steps, as well as derivatization.

To reveal such a trehalose receptor in mammals, the present inventors continued studying. As a result, they unexpectedly found the fact that a trehalose receptor is formed by combining a part of the receptor of sucrose with a G protein α-subunit, and also found that trehalose can be specifically and quantitatively detected using the trehalose receptor. Thus, they accomplished this invention.

The present invention solves the above object by providing a mammalian trehalose receptor and a method for detecting trehalose using the same.

### BRIEF DESCRIPTION OF THE ACCOMPANYING DRAWINGS

FIG. 1 shows the structure of a coexpression vector of G protein α-subunit α15 protein and α16/Z chimeric protein, according to the present invention.

FIG. 2 shows the structure of a T1R3 protein expression vector according to the present invention.

Explanation of symbols:

EF1 promoter: promotor of elongation factor
Gα15: G protein α-subunit α15 protein
poly A tail: poly (A) addition signal
Gα16/Z: G protein α-subunit α16/Z chimeric protein

### DETAILED DESCRIPTION OF THE INVENTION

The term “trehalose receptor” as referred to as in the present invention means a novel sweet-taste-receptor-combination formed on a cell membrane by expressing T1R3 protein (hereinafter abbreviated as “T1R3”, unless specified otherwise), as a sweet taste receptor, having the amino acid sequence of SEQ ID NO:5 in a cell which has been allowed to coexpress G protein α-subunits, α15, α16 and αZ proteins (hereinafter abbreviated as “α15”, “α16” and “αZ” respectively, unless specified otherwise), which have the amino acid sequences of SEQ ID NOs:1to 3, respectively; or a cell which has been allowed to coexpress α15 having the amino acid sequence of SEQ ID NO:1and G protein α-subunit α16/Z chimeric protein (hereinafter abbreviated as “α16/Z chimeric protein”, unless specified otherwise), having the amino acid sequence of SEQ ID NO:4, disclosed in Molecular Pharmacology, Vol. 57, pp. 13-23 (2000). These G protein α-subunits and T1R3 protein usable in the present invention include those which are derived from mammals independently of their species, or those each derived from different animal species. As the amino acid sequences of these proteins and the DNA sequences which encode them, those which are provided by gene data banks such as “GENEBANK”, produced by the National Institute of Health, USA. Particularly, T1R3 and α15 which are derived from mice, and α16 and αz which are derived from humans can be preferably used in view of their advantageous sensitivities. All of these proteins can be received a defect, replacement, or addition of an amino acid(s). These proteins can be expressed by coupling T1R3 with α15, α16, or α16/Z chimeric protein; or can be expressed respectively using a single vector. Since the α16/Z chimeric protein having SEQ ID NO:4 can be advantageously used in the present invention because it can coexpress α16 and αZ with a suitably reduced size of gene.

The cells used to express a trehalose receptor in the present invention include any cells as long as they form the trehalose receptor on their cell membrane surfaces and exhibit any reaction by binding to or reacting with trehalose. To improve the specificity of such a trehalose receptor to trehalose, preferably used are cells with no taste receptor other than cells with taste receptors such as taste cells. Particularly, 293 cells derived from human embryonic kidney epithelial cells, RCB 1637, available from the Riken Bioresource Center, Ibaraki, Japan, are preferably used in the present invention because they have no taste receptor and, as described later, the dynamics of intracellular calcium ion can be relatively easily detectable.

In practicing the method for expressing the trehalose receptor usable in the present invention, DNAs encoding the above-identified receptor proteins, for example, DNAs encoding the amino acid sequences of SEQ ID NOs:1 to 5 must be obtained. As the method for such obtention, those which chemically synthesize a part or the whole of the above-identified DNAs; those which selectively collect the desired DNAs from genomic DNAs, mRNAs, or cDNAs from animals using hybridization and PCR methods. An appropriate combination of these methods provides the DNAs requisite for practicing the present invention.

To express the trehalose receptor proteins, encoded by any of the above-identified DNAs, on the surface of cell membranes, such DNAs are introduced into appropriate expression vectors for animal cells and the resulting vectors are introduced/integrated into mammalian cells. Examples of such expression vectors include, usually, those which are used for animal cells and they can be appropriately selected. Any of the following can be used as such vectors; those which have an appropriate drug resistance gene, expression promotor region, polyadenylated site, polylinker, restriction enzyme cleavage site, or enhancer region; and other vectors such as plasmid-, virus-, and cosmid-vectors. In practicing the present invention, the expression for trehalose receptor protein may be a temporary or constant expression and can be selected to meet its purpose. The trehalose receptor protein can be also expressed in such a manner of introducing a DNA of any of the G proteins and a DNA encoding the trehalose receptor into different expression vectors, respectively; or introducing a DNA, which encodes a plurality of G proteins and the trehalose receptor, into a single expression vector.

The trehalose detection method of the present invention comprises the steps of adding a test sample, as a candidate, which may contain trehalose to animal cells in which the trehalose receptor, having a novel combination of a part of sucrose receptor and a part of a G protein α-subunit, has been expressed on the surface of the animal cell membranes; and detecting the biological reaction induced by the coupling of trehalose with the trehalose receptor. The above biological reaction includes reactions relating to intracellular signal transmission system. Using the reactions, methods for assaying the level of increase or decrease of the inflow of cyclic AMP, cyclic GMP, cyclic-nucleotide phosphodiesterase, protein kinase C, or calcium ion, which all relate to the above intracellular signal transmission system, can be employed. Particularly, among these methods, the one for assaying the inflow of calcium ion is advantageously used in the present invention because it is most easily practicable and is advantageous in sensitivity.

The method for assaying intracellular calcium ion usable in the present invention includes those which comprise the steps of reacting calcium ion with a reagent, which emits a fluorescence by coupling with calcium ion, for example, an reagent for detecting intracellular calcium ion such as “FLUO-4. AM”, a product name of Molecular Probes Inc., Ore., USA, to emit a fluorescence; and detecting the emitted fluorescence by using a commercialized plate-, cuvette-, or flow cytometric-fluorescence detector; or macroscopically observing the fluorescence by using a fluorescence microscope.

The trehalose detection method of the present invention specifically detects trehalose contained in food products and cosmetics as candidates. In the case of the candidates are in a solid, paste, gel, or lipophilic liquid form, they are prepared into test samples for detection after dissolving in aqueous solvents and removing insoluble substances. In the case of the candidates are in a hydrophilic liquid form, they can be assayed intact or after being dried into solids and then redissolved in an appropriate aqueous solvent. When the test samples are contaminated with impurities such as cytotoxic substances, minerals, and dyes/pigments which hinder the detection of trehalose, they can be optionally treated with appropriate separation methods such as absorption with activated charcoals, extraction with organic solvents, centrifugation, membrane filtration, gel filtration, ion-exchange chromatography, hydroxyapatite chromatography, hydrophobic chromatography, etc.; or treated with reagents such as acids, alkalis, reducing agents, oxidants, etc., to remove impurities. If necessary, the use of a sample as a negative control, which has been treated with trehalase as a trehalose hydrolase, will more accurately quantify trehalose. This method would be effective when the background of test samples is relatively high. The trehalose detection method of the present invention sensitively detects samples having a trehalose concentration of 5 to 50 mM. Before subjecting to the above method, test samples should be concentrated or diluted stepwisely to give their trehalose concentrations within the above range when the trehalose concentration of the test samples is outside the above range.

The trehalose detection method of the present invention is applicable to quantify the trehalose content in food products and cosmetics and to screen novel sweeteners in such a manner of evaluating the increased or decreased level of sweetening power of saccharides such as trehalose after derivatized.

The following Examples explain the present invention in detail:

### EXAMPLE 1

Construction of Vector for Expressing G Protein α-subunit

### EXAMPLE 1-1

Preparation of DNA Encoding G protein α-subunit α15

According to a conventional manner, RNAs containing mRNAs were extracted and purified from WEHI-3 cells, ATCC TIB-68, a mouse myelomonocytic leukemia cell line. One microgram of the resulting RNAs and 12.5 pmol of an appropriate random hexamer were reacted with “SUPER SCRIPT II RT”, a product name of Stratagene, CA, USA, at 42° C. for 50 min to synthesize a first strand cDNA. RNAs contaminated in the first strand cDNA were enzymatically hydrolyzed with ribonuclease I to obtain a template cDNA for PCR. A sense primer for PCR, having a nucleotide sequence of SEQ ID NO:7, was prepared by adding a nucleotide sequence having a cleavage site of restriction enzyme Hind III as a restriction enzyme to the 5′-terminus of a G protein α-subunit α15 DNA having the nucleotide sequence of SEQ ID NO:6; and an antisense primer for PCR, having the nucleotide sequence of SEQ ID NO:8, was prepared by adding a nucleotide sequence having a cleavage site of restriction enzyme Not I as a restriction enzyme to the 3′-terminus of the G protein α-subunit α15 DNA. Using “LA Taq DNA polymerase”, a thermostable DNA polymerase commercialized by Takara Shuzo Co., Ltd., Tokyo, Japan, the above cDNA and the primers for PCR were subjected to PCR in a usual manner to obtain a DNA encoding the G protein α-subunit α15.

### EXAMPLE 1-2

Preparation of DNA Encoding G Protein α16/Z Chimeric Protein

According to a conventional manner, RNAs containing mRNAs were extracted and purified from HL-60 cells, ATCC CCL 240, a promyelocytic cell line derived from a human with acute promyelocytic leukemia; and U-937 cell, ATCC CRL 1593.2, a human histocytic lymphoma cell line. A first strand cDNA was synthesized in a usual manner by allowing “SUPER SCRIPT II RT”, a reverse transcriptase commercialized by Stratagene, CA, USA, to act on one microgram of the above RNAs and 12.5 pmol of an appropriate random hexamer as a primer at 42° C. for 50 min. Concomitant RNAs were enzymatically hydrolyzed with ribonuclease I to obtain a cDNA as a template for PCR. To obtain a G protein α16 DNA having a nucleotide sequence of SEQ ID NO:9 and a G protein αZ DNA having a nucleotide sequence of SEQ ID NO:10, a sense primer for PCR, having a nucleotide sequence of SEQ ID NO:11, was prepared by adding a cleavage site of restriction enzyme Hind III to a DNA sequence around the initiation codon of α16, i.e., the 5′-terminus of the nucleotide residues 202 to 221 of the G protein α-subunit α16 DNA; and an antisense primer for PCR, having the nucleotide sequence of SEQ ID NO:12, was prepared by adding a complementary nucleotide sequence of the nucleotide residues 946 to 960 of the αZ DNA to the 5′-terminus of the nucleotide residues 1196 to 1211 of the α16 DNA. While, to obtain the G protein αZ DNA, there were prepared a sense primer for PCR, having the nucleotide sequence of SEQ ID NO:13, which had an additional nucleotide residues 1195 to 1211 of the α16 DNA at the 5′-terminus of the nucleotide residues 946 to 960 of SEQ ID NO:10; and an antisense primer, having the nucleotide sequence of SEQ ID NO:14, which had an additional cleavage site of restriction enzyme Not I at the 5′-terminus of nucleotide residues 1068 to 1086 of the G protein αZ DNA. Using “LA Taq DNA polymerase”, a thermostable DNA polymerase commercialized by Takara Shuzo Co., Ltd., Tokyo, Japan, the above cDNA and the primers for PCR were subjected to PCR in a usual manner in a prescribed combination to obtain a DNA encoding the G protein α-subunit α16 or αZ. The DNAs thus obtained were mixed, thermally denatured, allowed to anneal their overlapped parts, and subjected to PCR to obtain a DNA encoding an α16/Z chimeric protein with about 1,200 base pairs (bp).

### EXAMPLE 1-3

Construction of Vector Capable of Coexpressing G Protein α-subunit α15 Protein and G Protein α16/Z Chimeric Protein

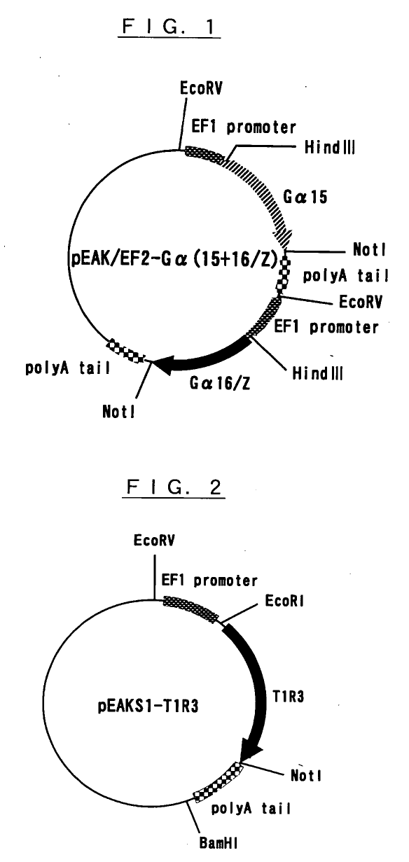

Using “pEAK12”, a plasmid vector commercialized by Edge BioSystems, MD, USA, as an expression vector, having a puromycin resistant gene, and an elongation factor 1α (EF-1α) promotor, etc., a cleavage site of restriction enzyme Eco RV was added to the restriction enzyme Spe I of the plasmid vector in a usual manner to obtain an expression vector pEAKS1, while a cleavage site of restriction enzyme Bam HI of the expression vector pEAKS1 was added in a usual manner to obtain an expression vector pEAKS2. The DNA encoding the G protein α-subunit α15 obtained in Example 1-1 and the DNA encoding the G protein α16/Z chimeric protein obtained in Example 1-2 were digested with restriction enzymes Hind III and Not I, respectively, and the resultants were ligated in a usual manner with the expression vectors pEAKS1 and pEAKS2 at their cleavage sites of Hind III and Not I, respectively, to insert a DNA encoding the G protein α-subunit α15 or α16/Z chimeric protein into the expression vectors pEAKS1 and pEAKS2, respectively. The resulting pEAKS2, into which the DNA encoding the G protein α16/Z chimeric protein had been introduced, was digested with a restriction enzyme Eco RV to obtain a DNA fragment containing a promoter region and a DNA sequence encoding the G protein α16/Z chimeric protein, followed by ligating the DNA fragment with the pEAKS1, having an inserted DNA encoding the G protein α-subunit α15, at its cleavage site of restriction enzyme Eco RV to obtain a coexpression vector, “pEAK/EF2-Gα (15 +16/Z)”, capable of coexpressing the G protein α-subunit α15 and the G protein α16/Z chimeric protein (cf. FIG. 1). Table 1 is a list of the PCR primers used in this experiment.

TABLE 1

| | GENBANK | | PCR primer | Sequence | |
|---|---|---|---|---|---|
| G Protein | accession number | Origin | 5′ 3′ | number | Remarks |
| α15 | M80632 | Mouse | CGCAAGCTT- | SEQ ID NO:7 | Hind III- |
| | | | TCTGTGAAGCGCCCACCATG | | α15 (26-45) |
| | | | GCATTACGATGCGGCCGC- | SEQ ID NO:8 | Not I- |
| | | | GCGTCACAGCAGGTTGATC | | α15 (1152-1170) |
| α16 | M63904 | Human | CGCAAGCTT- | SEQ ID NO:11 | Hind III- |
| | | | GACTGAGGCCACCGCACCAT | | α16 (202-221) |
| | | | CTCCTTGTTTCGGTT- | SEQ ID NO:12 | αZ (946-960)- |
| | | | GCTGCCCTCGGGGC | | α16 (1196-1211) |
| αZ | NM002073 | Human | GGCCCCGAGGGCAGC- | SEQ ID NO:13 | α16 (1195-1211) |
| | | | AACCGAAACAAGGAG | | αz (946-960) |
| | | | GCATTACGATGCGGCCGC- | SEQ ID NO:14 | Not I- |
| | | | AGCTCCTCAGCAAAGGCCA | | αZ (1068-1086) |

### EXAMPLE 2

Construction of Expression Vector of Mouse Sweet Taste Receptor Protein

### EXAMPLE 2-1

Preparation of DNAs for T1R1, T1R2 and T1R3

About 2.4 g of tongue tissues was collected from 16 wild type C57BL/6 mice, and RNAs including mRNAs were prepared therefrom. A first strand cDNA was synthesized in a usual manner by allowing “SUPER SCRIPT II RT”, a reverse transcriptase commercialized by Stratagene CA, USA, to act on one microgram of the above extracted RNAs and 12.5 pmol of an appropriate random hexamer as a primer at 42 ° C. for 50 min. According to conventional manner, the resulting RNAs were enzymatically hydrolyzed with ribonuclease I to obtain a cDNA as a template for PCR. To obtain DNAs for mouse sweet taste receptors T1R1, T1R2, and T1R3, having the nucleotide sequences of SEQ ID NOs:16, 17 and 18, respectively, a sense primer, having an additional cleavage site of restriction enzyme Eco RI at a nucleotide sequence around the initiation codon of each of the objective DNAs; and an antisense primer, having an additional cleavage site of restriction enzyme Not I at a complementary nucleotide sequence of the terminal codon of each of the objective DNAs, were prepared based on the DNAs for T1R1, T2R2and T1R3, registered at GENBANK. Using “LA Taq DNA polymerase”, a thermostable DNA polymerase commercialized by Takara Shuzo Co., Ltd., Tokyo, Japan, the above cDNAs and the primers for PCR were subjected to PCR in a usual manner in a prescribed combination to obtain a DNA encoding T1R1, T1R2, or T1R3, having a cleavage site of restriction enzyme Eco RI at its 5′-terminus and a cleavage site of restriction enzyme Not I at its 3′-terminus

### EXAMPLE 2-2

Construction of Expression Vector for Sweet Taste Receptor

As an expression vector for a sweet taste receptor, the expression vector “pEAKSN1” was prepared in a usual manner by replacing the puromycin resistant gene, as a drug resistant gene, of the expression vector “pEAKS1” in Example 1-3 with the expression vector “pREP9”, commercialized by Invitrogen, CA, USA, as a neomycin resistant gene. When expressing the desired sweet taste receptors respectively, each of the DNAs in Example 2-1 was digested with the restriction enzymes Eco RI and Not I and ligated in a usual manner with the expression vector pEAKSN1 at its restriction sites of Eco RI and Not I to obtain an expression vector for T1R1, T1R2 or T1R3 (cf. FIG. 2). When coexpressing the desired sweet taste receptors, a DNA for sweet taste receptor T1R1, T1R2 or T1R3 was introduced into the restriction site of Eco RI or Not I of the expression vector pEAKS2 to obtain an expression vector, followed by digesting the expression vector with a restriction enzyme Eco RV to obtain a DNA fragment including DNAs, encoding a promotor region and the sweet taste receptor protein. The DNA fragment was in a usual manner ligated with the cleavage site of restriction enzyme Eco RI of the expression vector pEAKSN1, including a DNA encoding another sweet taste receptor T1R1, T1R2 or T1R3 not contained in the expression vector pEAKS2, to obtain a coexpression vector for a pair of T1R1 and T1R2, T1R1 and T1R3, or T1R2 and T1R3. Table 2 is a list of the PCR primers used.

TABLE 2

| | GENBANK | | | | |
|---|---|---|---|---|---|
| Sweet taste | accession | | PCR primer | Sequence | |
| receptor | number | Origin | 5′ 3′ | number | Remarks |
| T1R1 | AY032622 | Mouse | GGAATTC- | SEQ ID NO:19 | Eco RI- |
| | | | ATGCTTTTCTGGGCAGCTCACC | | T1R1 (1-22) |
| | | | GCATTACGATGCGGCCGC- | SEQ ID NO:20 | Not I- |
| | | | TCAGGTAGTGCCGCAGCGCC | | T1R1 (2510-2529) |
| T1R2 | AY032623 | Mouse | GGAATTC- | SEQ ID NO:21 | Eco RI- |
| | | | ATGGGACCCCAGGCGAGGAC | | T1R2 (1-20) |
| | | | GCATTACGATGCGGCCGC- | SEQ ID NO:22 | Not I- |
| | | | CTAGCTCTTCCTCATCGTGTAG | | T1R2 (2511-2532) |
| T1R3 | AY032621 | Mouse | GGAATTC- | SEQ ID NO:23 | Eco RI- |
| | | | ATGCCAGCTTTGGCTATCATGG | | T1R3 (1-22) |
| | | | GCATTACGATGCGGCCGC- | SEQ ID NO:24 | Not I- |
| | | | TCATTCATTGTGTTCCTGAGCTG | | T1R3 (2555-2577) |

### EXAMPLE 3

Preparation of Cells Capable of Expressing Sweet Taste Receptor Proteins

The coexpression vector for the G protein α-subunit α15 and the G protein α16/Z chimeric protein (hereinafter designated as “G protein α-subunits”) obtained in Example 1-3 was gene transferred to 293 cells, RCB 1637, a human embryonic kidney cell line, available from the Riken Bioresource Center, Ibaraki, Japan, by conventional lipofection. The gene transferred cells were suspended in Dulbecco's Modified Eagle's Medium (D-MEM) supplemented with 1 mg/L of “PUROMYCIN”, a trade mane of puromycin commercialized by Edge Biosystems, MD, USA, and 10% (v/v) of fetal calf serum to give a cell density of 2×10⁶ cells/ml and cultured in a plastic petri dish for cell culture. After 10 to 14 days of incubation, a puromycin-resistant cell colony was collected and confirmed its production of the G protein α-subunit proteins with an index of their mRNA level by conventional RT-PCR. Thus, a cell line capable of expressing the G protein α-subunit proteins was established. To the cells were gene transferred an expression vector for the sweet taste receptor protein T1R1, T1R2, or T1R3 in Example 2-2; or a coexpression vector for a pair of T1R1 and T1R2, T1R1 and T1R3, or T1R2 and T1R3 by conventional lipofection. The resulting cells were suspended in D-MEM supplemented with 1 mg/L of “PUROMYCIN”, 500 mg/L of “GENETICIN”, and 10% (v/v) of fetal calf serum and cultured in a plastic petri dish for cell culture. After 10 to 14 days of incubation, a cell colony resistant to both of the above drugs was collected and confirmed that the transferred gene had been expressed intracellularly as expected by conventional RT-PCR at a mRNA level. Thus, a cell, which had coexpressed the desired G proteins and sweet taste receptors, was obtained. As a control, a cell, into which an expression vector with no G protein α-subunit protein gene or sweet taste receptor protein gene, was prepared and used.

### EXAMPLE 4

Reactivity Test on Trehalose and Sucrose Upon Sweet Taste Receptor

The captioned reactivity test was conducted according to a conventional intracellular calcium ion assay: 293 Cells prepared in Example 3, capable of coexpressing the G protein α-subunits and the sweet taste receptor(s), were cultured in a plastic petri dish for cell culture until reaching its confluent phase. Thereafter, the cells were detached from the inner surface of the petri dish with a 0.05% (v/v) trypsin solution and a 0.53 mM EDTA solution, suspended in D-MEM supplemented with 10% (v/v) fetal calf serum to give a cell density of 1×10⁶ cells/ml, and allowed to intake “FLUO-4. AM”, a reagent for detecting intracellular calcium ion commercialized by Molecular Probes Inc., Ore., USA, by adding to the cell suspension to give a final concentration of 2 μM and incubating the cells at 37 ° C. for 30 to 90 min. The resulting cells were washed with a buffer for detecting calcium ion, which contained 10 mM HEPES (pH 7.4), 130 mM sodium chloride, 5.4 mM potassium chloride, 2 mM calcium chloride, 1 mM magnesium chloride, 5.5 mM D-glucose, 0.1% (v/v) calf serum albumin, and 1 mM sodium pyruvate to remove the reagent remained extracellularly; suspended in a fresh preparation of the same buffer to give a cell density of 2.67 ×10⁷ cells/ml; filtered with a membrane with a pore size of 100 μm mesh; and allowed to stand at 25° C. for 30 min. Two milliliters of the resultant cell suspension was injected into a glass cuvette, commercialized by Hitachi, Ltd., Tokyo, Japan, and analyzed on “HITACHI 650-40”, a fluorescence spectrophotometer commercialized by Hitachi, Ltd., Tokyo, Japan.

Trehalose specimen as a test saccharide commercialized by Katayama Chemical, Co., Tokyo, Japan; and sucrose specimen, as a control, commercialized by Wako Pure Chemical Industries, Ltd., Tokyo, Japan, were respectively dissolved in the above buffer for calcium ion assay to give a concentration of 1 M. The resulting saccharide solutions were respectively placed in the above glass cuvette, containing the cell suspension, in a volume of 0.67 ml; stirred; and examined for reactivity between the saccharides and the cells by measuring the fluorescence intensity at an excitation wavelength of 494 nm and a fluorescence wavelength of 516 nm. The results are in Table 3.

TABLE 3

| Sweet taste receptor | | | | Influx of calcium ion | |
|---|---|---|---|---|---|
| T1R1 | T1R2 | T1R3 | G Protein α-subunit | Trehalose | Sucrose (control) |
| | — | — | | Negative | Negative |
| — | | — | | Negative | Negative |
| — | — | | | Positive | Negative |
| | | — | | Negative | Negative |
| | — | | | Positive | Negative |
| — | | | | Positive | Positive |
| — | — | — | | Negative | Negative |
| — | | | — | Negative | Negative |

As shown in Table 3, it was detected that the cells, which had coexpressed the G protein α-subunits and T1R3, reacted on trehalose. While sucrose as a control was detected with the cells which had expressed the G protein α-subunits and T1R2 and T1R3. These results revealed that the trehalose receptor does not require the sweet taste receptors T1R1 and T1R2 but the G protein α-subunits and the sweet taste receptor T1R3, and that trehalose and sucrose are recognized by different receptors in living bodies.

### EXAMPLE 5

Detection of Other Sweet Ingredients with Trehalose Receptor

The reactivity of the cells, which coexpressed the G protein α-subunits and the sweet taste receptor T1R3 in Example 4, on different sweet taste substances were assayed: The sweet taste substances listed in Table 4 were measured for influx of calcium ion similarly as in Example 4. The results are in Table 4.

TABLE 4

| Sweet taste substance | Influx of calcium ion |
|---|---|
| Trehalose | Positive |
| Sucrose | Negative |
| Mannose | Negative |
| Galactose | Negative |
| Fructose | Negative |
| Erythritol | Negative |
| Maltitol | Negative |
| L-Glycine | Negative |
| Alanine | Negative |
| Sucralose | Negative |
| Aspartame | Negative |

As shown in Table 4, the trehalose receptor has no reactivity to the saccharides other than trehalose, revealing that the receptor specifically recognizes trehalose. Thus, the trehalose receptor specifically detects trehalose even in a mixture form with various sweeteners.

### EXAMPLE 6

Quantitation of Trehalose using Trehalose Receptor

One hundred microliter aliquots of the cell suspension in Example 4 were placed in commercially available 96-well microplates, followed by placing 0.1 ml/well of any of trehalose solutions with different concentrations of 1, 2, 5, 10, 20, 50, 100, 200, 500, 1,000, and 2,000 mM, prepared with trehalose and the same buffer for calcium ion assay as used in Example 4; and assayed on “FLUOROSCAN ASCENT W/DF”, an automatic fluorescence spectrophotometer for multi-plates, Dainippon Pharmaceutical Co., Ltd., Tokyo, Japan, at an excitation wavelength of 494 nm and a fluorescence wavelength of 516 nm, followed by calculating the integral values for the detected fluorescence intensities. The results are in Table 5.

TABLE 5

| Concentration of trehalose | Fluorescence intensity |
|---|---|
| (mM) | (integral value) |
| 0 | 0 |
| 1 | 0.2 |
| 2 | 0.5 |
| 5 | 10 |
| 10 | 19 |
| 20 | 29 |
| 50 | 45 |
| 100 | 82 |
| 200 | 159 |
| 500 | 421 |
| 1,000 | 670 |
| 2,000 | 720 |

As shown in Table 5, trehalose is detectable at concentrations of 5 mM or over and is detectable up to a concentration of 500 mM in a direct proportional manner. These results show that the assay with the trehalose receptor quantitatively detects trehalose at concentrations in the range of 5 to 500 mM.

Thus, the present invention facilitates the detection and quantitation of trehalose present in vivo and in vitro quite accurately and easily even if trehalose in a test sample is contaminated with other saccharides such as sucrose because of the use of receptor specific to trehalose according to the present invention.

1. A mammalian trehalose receptor, comprising:
(i) a protein comprising any of the amino acid sequences of SEQ ID NOs: 1, 2, 3 and 5; or a protein comprising any of the amino acid sequences thereof with a deletion, replacement, or addition of an amino acid(s); or
(ii) a protein comprising any of the amino acid sequences of SEQ ID NOs: 1, 4 and 5; or a protein comprising any of the amino acid sequences thereof with a deletion, replacement, or addition of an amino acid(s).

2. An animal cell, comprising an artificially expressed trehalose receptor of claim 1.

3. A process for producing an animal cell comprising an artificially expressed trehalose receptor, said process comprising a step of introducing an expression vector comprising an integrated DNA into an animal cell, said DNA encoding:
(i) a protein comprising any of the amino acid sequences of SEQ ID NOs: 1, 2, 3 and 5; or a protein comprising any of the amino acid sequences thereof with a deletion, replacement, or addition of an amino acid(s); or
(ii) a protein comprising any of the amino acid sequences of SEQ ID NOs: 1, 4 and 5, or a protein comprising any of the amino acid sequences thereof with a deletion, replacement, or addition of an amino acid(s).

4. A method for detecting trehalose using an animal cell comprising an artificially expressed trehalose receptor of claim 1 or 2.

5. The method of claim 4, which detects a biochemical reaction induced by the binding of trehalose to said trehalose receptor.

6. The method of claim 5, wherein said biochemical reaction is detected by measuring the influx of calcium ion.

7. A kit for detecting trehalose, comprising the animal cell of claim 2 and a reagent for detecting calcium ion.
